(19) United States
(12) Patent Application Publication
(10) Pub. No.: US 2026/0351423 A1
(43) Pub. Date: Nov. 5, 2026

(54) METHOD AND SYSTEM APPARATUS FOR EXTRACTING, STANDARDIZING, INTEGRATING, AND EXCHANGING MEDICAL DATA FROM FREE-TEXT-BASED REAL-WORLD CANCER DATA USING AN AI SYSTEM

(71) Applicant: SEOUL MEDICAL INFORMATICS INTELLIGENCE LAB INC., Seoul (KR)

(72) Inventors: Sihyun SUNG, Seoul (KR); Ye-lim CHOI, Seoul (KR); Eun-Jung YANG, Seoul (KR)

(21) Appl. No.: 19/403,436

(22) Filed: Nov. 28, 2025

(30) Foreign Application Priority Data
Apr. 15, 2025 (KR) 10-2025-0048966

(51) Int. Cl. G16H 50/70; G06N 5/022; G16H 10/60

(52) U.S. Cl. CPC G16H 50/70; G06N 5/022; G16H 10/60

(57) ABSTRACT

A data extraction method performed by a data extraction system comprises: building a knowledge graph by linking a data dictionary of medical data according to contextual information, wherein the data dictionary refers to data organized according to a medical standard format; and analyzing unstructured medical data related to a patient by using a Large Language Model (LLM) and the constructed knowledge graph, thereby extracting clinical data related to a user from the unstructured medical data.

### CROSS-REFERENCE TO RELATED APPLICATION

This application is based on and claims priority under 35 U.S.C. § 119 to Korean Patent Application No. 10-2025-0048966, filed on Apr. 15, 2025, in the Korean Intellectual Property Office, the disclosure of which is incorporated by reference herein in its entirety.

### BACKGROUND

### Field

The present invention relates to a technology for extracting, standardizing, integrating, and exchanging medical data.

A Large Language Model (LLM) has been attracting attention as a core technology that expands the scope of data analysis and enhances the efficiency and accuracy of data-driven decision-making. Beyond merely generating text, it demonstrates excellent performance in various natural language processing (NLP) tasks such as translation, summarization, question answering, and sentiment analysis.

As artificial intelligence (AI) technology has advanced, there has been an increasing demand for analyzing unstructured medical data to derive valuable outcomes. Such unstructured medical data needs to be refined into a format suitable for the intended service.

### SUMMARY

It is possible to provide a method and system for extracting clinical data usable for new drug development and clinical research by utilizing a large language model from unstructured or structured medical data stored in formats such as Excel, text, and image files.

It is also possible to provide a method and system for delivering the extracted clinical data to users in compliance with Fast Healthcare Interoperability Resources (FHIR).

A clinical data extraction method performed by a data extraction system may include: building a knowledge graph by linking a data dictionary of medical data according to contextual information, wherein the data dictionary refers to a dataset organized according to a medical standard format; and analyzing unstructured medical data related to a patient by using the knowledge graph built through a Large Language Model (LLM), thereby extracting clinical data related to a user from the unstructured medical data.

The building step may include constructing a knowledge graph composed of a head entity, a tail entity, and a relation with respect to the medical data.

The head entity may include health information including at least one of a diagnosis name, a treatment method, a symptom, and a test name based on a value set represented by code information defined in the data dictionary of the medical data. The tail entity may include auxiliary information describing the health information. The relation may include information indicating a semantic connection between the head entity and the tail entity.

The clinical data extraction method may further include providing a user interface that displays multiple items of the knowledge graph through a knowledge graph search tool and allows a user to select at least one item from among the displayed items in order to select medical data to be extracted, where each item includes the head entity, the tail entity, and the relation, and the selected item may be used to construct a prompt to be input to the large language model for extracting the clinical data.

The large language model may refer to the selected item of the knowledge graph so that the clinical data is extracted from the unstructured medical data.

The unstructured medical data may include unstructured text of an electronic medical record (EMR). The prompt may include at least a portion of the unstructured text and the selected item. As the prompt is input to the large language model, the clinical data may be extracted from the unstructured medical data. The large language model may operate in a Retrieval-Augmented Generation (RAG) or Retrieval-Augmented Fine-Tuning (RAFT) manner.

The extracting step may include converting the extracted clinical data based on an FHIR (Fast Healthcare Interoperability Resources) structure and storing the clinical data converted into the FHIR structure in an FHIR server.

The extracting step may further include storing data related to the selected item of the knowledge graph in a database.

The clinical data extraction method may further include embedding the constructed knowledge graph and using the embedded graph for fine-tuning of the large language model.

The embedding may represent semantic distances among medical concepts as vectors by using at least one algorithm among Translation Embedding (TransE), Graph Convolutional Network (GCN), and Graph Attention Network (GAT). The represented vectors may be used as input to, or for fine-tuning of, the large language model.

The clinical data extraction method may further include building a database including abbreviations, typos, synonyms, and similar expressions of keywords contained in the data dictionary of the medical data, and constructing an extended knowledge graph based on the built database. The extended knowledge graph may be utilized by the large language model for the extraction of clinical data as the knowledge graph.

By constructing a knowledge graph for structured or unstructured medical data, it is possible to improve the accuracy of clinical data extraction.

By building a knowledge graph including abbreviations, synonyms, and similar expressions of keywords in the data dictionary of medical data, it is possible to enhance the performance of the large language model.

### BRIEF DESCRIPTION OF THE DRAWINGS

FIG. 1 is a diagram illustrating a clinical data extraction operation according to an embodiment of the present invention.

FIG. 2 is a diagram illustrating a knowledge graph according to an embodiment of the present invention.

FIG. 3 is a diagram illustrating a knowledge graph search tool according to an embodiment of the present invention.

FIG. 4 is a block diagram illustrating a data extraction system according to an embodiment of the present invention.

FIG. 5 is a flowchart illustrating a data extraction method according to an embodiment of the present invention.

### DETAILED DESCRIPTION

Hereinafter, embodiments of the present invention will be described in detail with reference to the accompanying drawings.

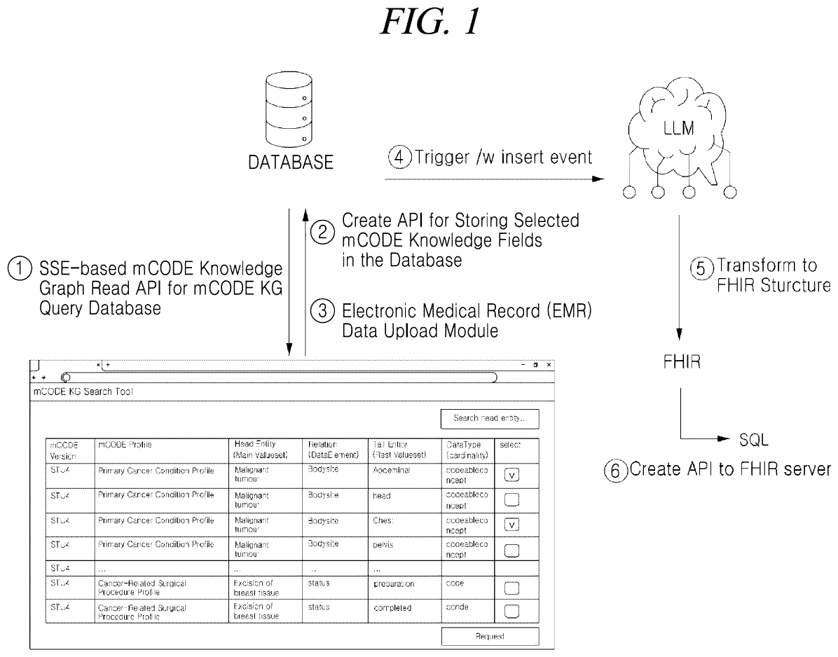

FIG. 1 is a diagram illustrating a clinical data extraction operation according to an embodiment of the present invention.

A data extraction system may build a knowledge graph by linking a data dictionary of medical data according to contextual information. In an embodiment, minimal common oncology data elements (mCODE) are described as an example of medical data for clinical data extraction in cancer research. Here, the data dictionary refers to a dataset organized according to a medical standard format, listing elements of medical data and detailed information on the respective elements. In this case, the data dictionary may be an mCODE (Minimal Common Oncology Data Elements) data dictionary. The mCODE is a standardized data model for essential medical data used in cancer patient treatment and research, and may be configured with structured data such as tumor diagnosis information, cancer stage, treatment history, and genomic information.

The data extraction system may analyze unstructured medical data related to a patient by using a knowledge graph constructed through a Large Language Model (LLM), thereby extracting clinical data related to a user from the unstructured medical data. For example, a Generative Pre-trained Transformer (GPT) may be used as the large language model. The large language model may be pre-trained with the data dictionary of medical data or additionally trained with the constructed knowledge graph.

The data extraction system may display multiple items of the knowledge graph through a knowledge graph search tool. The data extraction system may retrieve the knowledge graph through a Read API for SSE (Server-Sent Events)-based knowledge graph query (1). The data extraction system may provide a user interface that allows selection of at least one item from among the multiple displayed items of the knowledge graph to select medical data to be extracted. Each item may include a head entity, a tail entity, and a relation.

The data extraction system may store data related to the selected item of the knowledge graph in a database. The data extraction system may store data related to the selected item in the database through a Create API by a clinical researcher (2).

The electronic medical record (EMR) may be uploaded by a clinical researcher (3). The data extraction system may collect unstructured medical data related to a patient uploaded by the clinical researcher. The unstructured medical data may include unstructured text of an electronic medical record (EMR). The unstructured medical data may include patient medical information composed of text written without a specific format. For example, the unstructured medical data may include free-form text data containing various types of medical information stored in an unstructured form. In one example, the unstructured medical data may include documents written in unstructured text such as a physician's clinical note, a nurse's progress note, a radiology report, a surgical or treatment record, and a pathology report, and may include text containing more detailed and contextual information such as a patient's symptoms, disease progression, and treatment response. In other words, the unstructured medical data may include information necessary to understand a patient's overall health condition and treatment history, while being unstructured data.

As data related to the selected item of the knowledge graph is stored in the database, a specific trigger event may occur to execute a prompt generation operation (4). The data extraction system may input the selected item by the clinical researcher and the unstructured text of the electronic medical record as request parameters to a framework API.

The data extraction system may generate a prompt for clinical data extraction by using the data related to the selected item of the knowledge graph and the unstructured text of the electronic medical record. For example, the data extraction system may generate a prompt for clinical data extraction by using the data related to the selected item of the knowledge graph and the unstructured text of the electronic medical record according to a predefined template for clinical data extraction. The prompt may be configured to reflect types of clinical entities to be extracted, related symptoms, diagnosis names, medication information, or temporal information. In addition, the prompt may be designed to include relational information among multiple entities linked on the knowledge graph, thereby enabling more accurate and context-aware data extraction. For instance, the prompt may be generated as a natural language-based prompt such as “Summarize the medication history related to Disease A when the patient's diagnosis is Disease A,” where the components of the prompt may be determined from the medication nodes linked to “Diagnosis: Disease A” in the knowledge graph and relevant sentences within the electronic medical record.

The large language model may operate in a Retrieval-Augmented Generation (RAG) or Retrieval-Augmented Fine-Tuning (RAFT) manner. For example, the knowledge graph may be applied to a RAG architecture based on a large language model. In other words, the data extraction system may search contextual information for a user query from the knowledge graph and include the retrieved knowledge graph as input to the large language model, thereby ensuring the accuracy and consistency of responses.

As the prompt is input to the large language model, clinical data may be extracted from the unstructured medical data. Such clinical data may be extracted in a structured table format (for example, JSON or CSV file format) and may be converted into various file formats (for example, CDM, XML, or RDF) according to a user's request. The data extraction system may convert the extracted clinical data into a Fast Healthcare Interoperability Resources (FHIR) structure (5). The FHIR is a standard framework developed by the Health Level Seven (HL7) organization to promote interoperability of healthcare information. In this process, the data extraction system may utilize the large language model to convert the extracted clinical data into the FHIR structure. The data extraction system may prepare the FHIR-structured clinical data for linkage with an FHIR server. The data extraction system may store the clinical data converted into the FHIR structure in an SQL-based (for example, PostgreSQL) FHIR server through a Create API (6). Through this process, the clinical researcher may retrieve desired clinical data based on the FHIR structure via SQL queries.

In addition, the data extraction system may embed the knowledge graph and use the embedded knowledge graph for fine-tuning of the large language model for cancer research. The data extraction system may fine-tune the large language model in a Retrieval-Augmented Fine-Tuning (RAFT) manner through embedding of the knowledge graph. Accordingly, a structure capable of storing specific domain knowledge as long-term memory may be realized.

In this case, the data extraction system may embed the knowledge graph by using a graph neural network (GNN)-based algorithm. The data extraction system may represent semantic distances between medical concepts as vectors by using at least one algorithm among Translation Embedding (TransE), Graph Convolutional Network (GCN), and Graph Attention Network (GAT), and may utilize the represented vectors as input to, or for fine-tuning of, the large language model. For example, each node (for instance, diagnosis name, test name, or symptom) and relation (for instance, test result or affected body part) may be represented in a low-dimensional embedding space through vectorization techniques such as TransE (Translation Embedding), GCN (Graph Convolutional Network), GAT (Graph Attention Network), or ComplEx. These vectors preserve semantic similarity and structural relationships among medical concepts and may enhance the contextual understanding capability of the large language model in extracting meaningful information from unstructured texts of medical data (for example, clinical documents).

Furthermore, the data extraction system may provide or utilize knowledge graph-based domain knowledge for the large language model in real time based on a Retrieval-Augmented Generation (RAG) or Retrieval-Augmented Fine-Tuning (RAFT) architecture. In the RAG method, related information may be retrieved from the knowledge graph during query processing, and the retrieved information may be provided to the large language model together with the prompt. In the RAFT method, a training dataset including knowledge graph embeddings may be constructed to perform domain-specific learning of the large language model. Through this, the accuracy and reproducibility of clinical data extraction may be improved.

In addition, the data extraction system may construct a dataset using the embedded knowledge graph and utilize it for training of the large language model. The embedded knowledge graph may be used in the input or fine-tuning process of the large language model by reflecting relationship information among medical data. For example, each node (h, t) and relation (r) may be vectorized in a ternary structure (h+r≈t) through Translation Embedding (TransE), and structural information of the relational network may be reflected in the embedding through GCN or GAT. The generated vectors may be used as background knowledge in RAG query contexts or RAFT training texts of the large language model. Since the medical data may include different keywords for the same condition (for example, diagnosis name) depending on the medical standard, usage environment, or medical staff, the data extraction system may build a database including abbreviations, typos, synonyms, and equivalent expressions of keywords contained in the data dictionary of the medical data, and may construct an extended knowledge graph based on the built database. The extended knowledge graph may be utilized by the large language model as the knowledge graph for clinical data extraction. In addition, the data extraction system may embed the extended knowledge graph and apply it to a Retrieval-Augmented Generation (RAG) structure for use in development of a Retrieval-Augmented Fine-Tuning (RAFT) model, so that the large language model can more efficiently utilize specific domain knowledge.

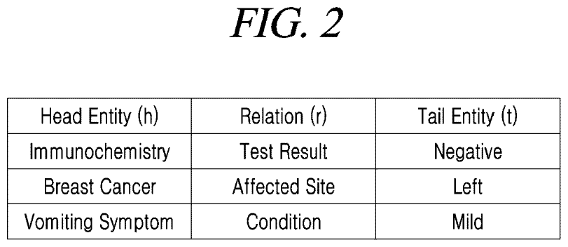

FIG. 2 is a diagram illustrating a knowledge graph according to an embodiment of the present invention.

The data extraction system may build a knowledge graph by linking a data dictionary of medical data according to contextual information. The knowledge graph may be provided to the large language model in the form of a training dataset for real-time retrieval or learning, thereby improving the accuracy of question answering and the performance of clinical information extraction in the medical domain. In other words, the data extraction system may reclassify a value set defined in the data dictionary of medical data into a head entity (h), a tail entity (t), and a relation (r), and may build a knowledge graph by applying the reclassified head entity, tail entity, and relation to a hierarchical knowledge graph structure. Here, the value set refers to the value stored in the medical data.

The head entity refers to data stored in a higher-level node, describing the main concept among the value sets. The head entity is a basic main concept defined in a medical terminology ontology, and may represent, for example, a diagnosis name, treatment method, test name, or symptom. Typically, value sets expressed by code information in SNOMED CT or LOINC are used. The main concept represents a core element that expresses health information and constitutes the overall structure by including other hierarchical concepts. Generally, each profile of medical data has its own value set. The profile may be an mCODE profile. The profile may represent a concept higher than the head entity.

The tail entity refers to data stored in a lower-level node and has a dependent relationship with the main concept among the value sets, providing auxiliary information describing the main concept. The supporting concept provides detailed and specific information that complements the main concept, reflecting an individual patient's condition or characteristics. It provides detailed explanations necessary to express medical data in a patient-customized manner and may be utilized in various domains such as diagnosis, examination, and treatment.

The relation represents the subject of the dependent relationship between the head entity and the tail entity. The relational concept provides a semantic linkage between the main concept representing health information (for example, disease name, examination name, treatment name, etc.) and the supporting concept that describes or extends the health information.

Such a knowledge graph may be composed of multiple items. The number of items may be a predetermined number (for example, 50). In other words, the knowledge graph may be composed of 50 pairs of head entity-relation-tail entity combinations. Alternatively, there may be 50 types of head entities. In this case, even if the head entity is the same, multiple items having different relations and tail entities may exist.

Referring to the example illustrated in FIG. 2, when the main concept is “immunochemistry test” and the supporting concept is “negative,” the relational concept becomes “test result.” When the main concept is “breast cancer” and the supporting concept is “left,” the relational concept becomes “affected site.” Similarly, when the main concept is “vomiting symptom” and the supporting concept is “mild,” the relational concept becomes “condition.”

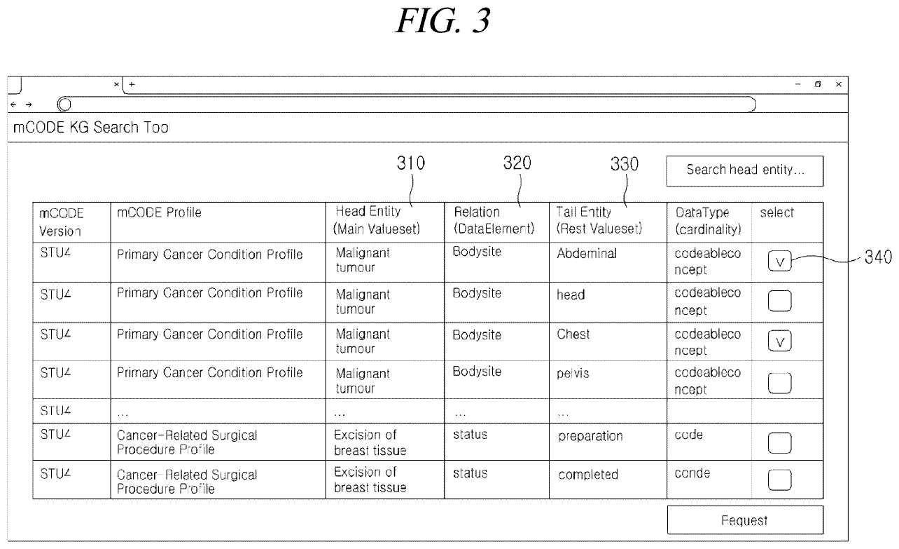

FIG. 3 is a diagram illustrating a knowledge graph search tool according to an embodiment of the present invention.

The data extraction system may display multiple items of a constructed knowledge graph through a knowledge graph search tool. The data extraction system may provide a user interface that allows a clinical researcher to search, view, and select within the constructed knowledge graph in order to accurately define the medical data to be extracted. For example, the knowledge graph search tool may provide information such as a medical data version, medical data profile, head entity (310), relation (320), tail entity (330), data type, and user interface (340).

The data extraction system may provide a user interface (340) that allows the user to select at least one item from among the multiple items in order to specify the medical data to be extracted. The data extraction system may receive a selection of one or more items among the multiple items of the knowledge graph through the user interface (340) provided by the knowledge graph search tool and may reference the selected items. The selected item may be used to construct a prompt that is input to the large language model for clinical data extraction. When the unstructured medical data is input to the large language model, the large language model may analyze the text of the unstructured medical data and extract meaningful clinical data such as patient conditions, disease diagnoses, and treatment information. At this time, the knowledge graph may be used in a “reference” manner together with the large language model to enable the model to extract more accurate and consistent information.

The knowledge graph may be a data structure that represents structured knowledge of the medical domain-such as medical terminology, relationships between diseases, drug information, and clinical guidelines-in the form of nodes and edges. By providing the knowledge graph information together with the prompt, the large language model can link specific expressions or terms in the input unstructured text (unstructured medical data) with concepts in the graph, thereby interpreting their meaning clearly and assisting in making accurate clinical judgments in context. For example, when the large language model analyzes a sentence such as “The patient was diagnosed with HER2-positive breast cancer and started trastuzumab treatment,” the model can refer to the relationships among “HER2,” “breast cancer,” and “trastuzumab” in the knowledge graph to understand the patient's pathological state and treatment strategy more precisely. Through such referencing, the large language model can not only comprehend the text contained in the unstructured medical data but also extract valid clinical data therefrom.

Here, the unstructured medical data includes unstructured text of electronic medical records (EMRs). The data extraction system may collect electronic medical records uploaded by a user (for example, a clinical researcher). The data extraction system may provide a user interface for uploading electronic medical records. Using the user interface, a clinical researcher may upload electronic medical record files in specific formats (for example, Excel, CSV, JPG, or PDF). For instance, electronic medical records may include documents written in text form such as pathology reports, imaging interpretation reports, progress notes, and discharge summaries. The data extraction system may perform preprocessing to convert the uploaded electronic medical records into a form suitable for data extraction. In addition, through the user interface for electronic medical record upload, the data extraction system may modify, delete, or add uploaded electronic medical records.

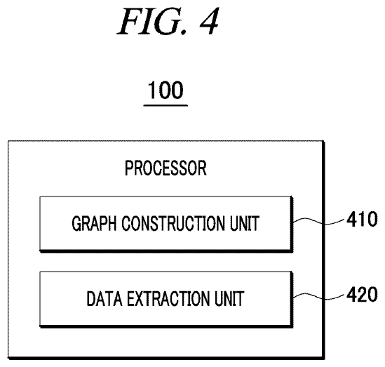

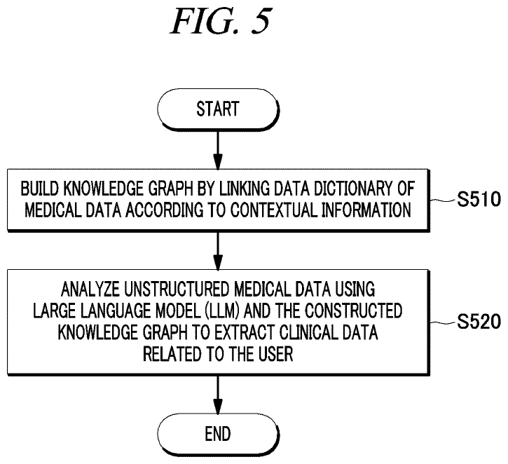

FIG. 4 is a block diagram illustrating a data extraction system according to an embodiment of the present invention, and FIG. 5 is a flowchart illustrating a data extraction method according to an embodiment of the present invention.

The processor (100) of the data extraction system may include a graph construction unit (410) and a data extraction unit (420). The components of the processor may represent different functions performed by the processor according to control commands provided by program code stored in the data extraction system. The processor and its components may control the data extraction system to perform the steps (510 to 520) included in the data extraction method illustrated in FIG. 5. In this case, the processor and its components may be implemented to execute instructions according to the code of an operating system and at least one program stored in memory.

The processor may load program code stored in a program file for the data extraction method into memory. For example, when the program is executed in the data extraction system, the processor may control the system to load the program code from the program file into memory under control of the operating system. The graph construction unit (410) and the data extraction unit (420) may each execute corresponding portions of the program code loaded in memory, serving as different functional representations of the processor for performing subsequent steps (510 to 520).

In step (510), the graph construction unit (410) may build a knowledge graph by linking a data dictionary of medical data according to contextual information. The graph construction unit (410) may build the medical data as a knowledge graph composed of a head entity, a tail entity, and a relation. The head entity may include health information comprising at least one of a diagnosis name, treatment method, symptom, and test name based on a value set represented by code information defined in the data dictionary of the medical data; the tail entity may include auxiliary information describing the health information; and the relation may include information indicating a semantic connection between the head entity and the tail entity. The graph construction unit (410) may display multiple items of the constructed knowledge graph through a knowledge graph search tool and may provide a user interface that allows selection of at least one item from among the multiple items in order to select medical data to be extracted. Each item may include a head entity, a tail entity, and a relation, and the selected item may be used to construct a prompt input to the large language model for extracting clinical data.

In step (520), the data extraction unit (420) may analyze unstructured medical data related to a patient by using a large language model (LLM) and the constructed knowledge graph, thereby extracting clinical data related to a user from the unstructured medical data. The selected item of the knowledge graph may be referenced by the large language model so that the clinical data is extracted from the unstructured medical data. The unstructured medical data may include unstructured text of an electronic medical record (EMR). The prompt may include at least a portion of the unstructured text and the selected item. As the prompt is input to the large language model, the clinical data may be extracted from the unstructured medical data. The data extraction unit (420) may convert the extracted clinical data based on a Fast Healthcare Interoperability Resources (FHIR) structure and may store the clinical data converted into the FHIR structure in an FHIR server. The data extraction unit (420) may also store data related to the selected item of the knowledge graph in a database.

The apparatus described above may be implemented using hardware components, software components, and/or a combination of hardware and software components. For example, the devices and components described in the embodiments may be implemented using one or more general-purpose or special-purpose computers, such as a processor, controller, arithmetic logic unit (ALU), digital signal processor (DSP), microcomputer, field programmable gate array (FPGA), programmable logic unit (PLU), microprocessor, or any other device capable of executing and responding to instructions. The processing device may execute an operating system (OS) and one or more software applications running on the operating system. The processing device may access, store, manipulate, process, and generate data in response to execution of software. For convenience of description, a single processing device is described; however, it will be understood by those skilled in the art that the processing device may include a plurality of processing elements and/or a plurality of types of processing elements. For example, the processing device may include multiple processors, or a combination of a processor and a controller. Other processing configurations, such as a parallel processor configuration, may also be possible.

Software may include a computer program, code, instruction, or a combination thereof, and may configure or independently and collectively command the processing device to operate as desired. The software and/or data may be embodied in any type of machine, component, physical device, virtual equipment, computer storage medium, or device to be interpreted by or to provide instructions or data to the processing device. The software may also be distributed over networked computer systems and stored or executed in a distributed manner. The software and data may be stored on one or more computer-readable recording media.

The method according to an embodiment may be implemented in the form of program instructions executable through various computer means and recorded on a computer-readable medium. The computer-readable medium may include program instructions, data files, and data structures individually or in combination. The program instructions recorded on the medium may be specifically designed and configured for the embodiment, or may be known and available to those skilled in computer software. Examples of computer-readable recording media include magnetic media such as hard disks, floppy disks, and magnetic tapes; optical media such as CD-ROMs and DVDs; magneto-optical media such as floptical disks; and hardware devices specifically configured to store and execute program instructions such as ROM, RAM, and flash memory. Examples of program instructions include not only machine code generated by compilers but also high-level language code executable by computers using interpreters and the like.

Although the embodiments have been described with reference to limited examples and drawings, it will be understood by those skilled in the art that various modifications and variations can be made from the above description. For example, the described technologies may be performed in a sequence different from that described, and/or the components of the described systems, structures, devices, or circuits may be combined or configured in different forms, or replaced or substituted with other components or equivalents, without departing from the scope of the present invention.

Therefore, other implementations, variations, and equivalents are also within the scope of the following claims.

What is claimed is:

1. A clinical data extraction method performed by a data extraction system, the method comprising:
building a knowledge graph by linking a data dictionary of medical data according to contextual information,
wherein the data dictionary refers to data organized according to a medical standard format; and
analyzing unstructured medical data related to a patient by using a Large Language Model (LLM) and the constructed knowledge graph,
thereby extracting clinical data related to a user from the unstructured medical data.

2. The clinical data extraction method according to claim 1,
wherein the building step comprises constructing the medical data as a knowledge graph composed of a head entity, a tail entity, and a relation,
wherein the head entity includes health information comprising at least one of a diagnosis name, a treatment method, a symptom, and a test name based on a value set represented by code information defined in the data dictionary of the medical data,
the tail entity includes auxiliary information describing the health information, and
the relation includes information indicating a semantic connection between the head entity and the tail entity.

3. The clinical data extraction method according to claim 2,
further comprising displaying multiple items of the constructed knowledge graph through a knowledge graph search tool and providing a user interface that allows selection of at least one item from among the multiple items to select medical data to be extracted,
wherein each of the items includes the head entity, the tail entity, and the relation, and
the selected item is used to construct a prompt input to the LLM for extracting the clinical data.

4. The clinical data extraction method according to claim 3,
wherein the selected item of the knowledge graph is referenced by the LLM so that the clinical data is extracted from the unstructured medical data.

5. The clinical data extraction method according to claim 4,
wherein the unstructured medical data includes unstructured text of an Electronic Medical Record (EMR),
wherein the prompt comprises at least a portion of the unstructured text and the selected item,
and as the prompt is input to the LLM, the clinical data is extracted from the unstructured medical data, and the Large Language Model operates in a Retrieval-Augmented Generation (RAG) or Retrieval-Augmented Fine-Tuning (RAFT) manner.

6. The clinical data extraction method according to claim 5,
wherein the extracting step comprises converting the extracted clinical data based on a Fast Healthcare Interoperability Resources (FHIR) structure and storing the clinical data converted into the FHIR structure in an FHIR server.

7. The clinical data extraction method according to claim 4,
wherein the extracting step comprises storing data related to the selected item of the knowledge graph in a database.

8. The clinical data extraction method according to claim 1,
further comprising embedding the constructed knowledge graph and using the embedded knowledge graph for fine-tuning of the LLM.

9. The clinical data extraction method according to claim 8,
wherein the embedding represents semantic distances between medical concepts as vectors by using at least one algorithm among Translation Embedding (TransE), Graph Convolutional Network (GCN), and Graph Attention Network (GAT),
and the represented vectors are utilized as input to, or for fine-tuning of, the LLM.

10. The clinical data extraction method according to claim 1,
further comprising building a database including abbreviations, typos, synonyms, and equivalent expressions of keywords contained in the data dictionary of the medical data, and constructing an extended knowledge graph based on the built database,
wherein the extended knowledge graph is utilized as the knowledge graph by the LLM for extraction of the clinical data.
